(19) United States
(12) Patent Application Publication
(10) Pub. No.: US 2023/0139953 A1
(43) Pub. Date: May 4, 2023

(54) SYSTEM AND METHOD FOR MONITORING THE PERFUSION OF A TISSUE

(71) Applicant: VYGON, Ecouen (FR)

(72) Inventors: Bernard ALLAOUCHICHE, Lyon (FR); Benjamin DELCROIX, Villeneuve-Loubet (FR); Dominique FALCON, Lyon (FR); Remy BONIDAL, Villeurbanne (FR)

(21) Appl. No.: 16/647,797

(22) PCT Filed: Jun. 28, 2018
(86) PCT No.: PCT/FR2018/051583

§ 371 Date: Mar. 16, 2020

(30) Foreign Application Priority Data
Jun. 30, 2017 (FR) 1700698

(51) Int. Cl. A61B 5/00; A61B 5/20

(52) U.S. Cl. CPC A61B 5/0084; A61B 5/202; A61B 5/6853

(57) ABSTRACT

The system for monitoring the perfusion of a tissue includes a perfusion sensor and a pressure sensor disposed near or at the perfusion sensor. The sensors are paired with a treatment unit. The treatment unit includes at least one correcting module to determine and/or correct the artifacts of the perfusion sensor linked to a pressure measured by the pressure sensor. The invention further relates to a corresponding monitoring method.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

This invention regards the domain of measurement devices and systems to estimate the metabolic parameters regarding the perfusion of a patient's organ, particularly an organ with a mucous membrane and muscular tissues. The invention furthermore includes a related monitoring procedure.

Such a system could be used in several applications for monitoring patients prone to tissue hypo-perfusion. These disorders can have various origins: hemorrhagic, infectious or inflammatory.

### 2. Description of Related Art Including Information Disclosed Under 37 CFR 1.97 and 37 CFR 1.98

Monitoring the biological perfusion parameters makes it possible to use information regarding the organ's microcirculation to help in the choice of treatments adapted to the patient. Indeed, good distal perfusion of the organs of at-risk patients is an important element for treating them. Bad perfusion of the organs can cause major problems, even causing the failure of these organs.

One of the difficulties of surveying distal perfusion is that it can be heterogenic and, in certain cases, it can be enough in one place and insufficient in another. When there are changes in blood flow, micro-circulation can be affected.

According to scientific literature, urethral mucous seems to be particularly sensitive to hemodynamic changes. Therefore, it is particularly interesting for monitoring the organism's response to therapeutic actions. Therapeutic actions are typically done on the whole organism and have an effect on each of the organs.

Knowing the state of local perfusion and urethral mucous is an important element for treating patients that may have alterations on this perfusion.

For example, photo-plethysmography technology is a way to evaluate the perfusion of a tissue. Generally, a catheter equipped with a photo-plethysmography sensor is used. Unfortunately, regarding particular tissue like those from the urethra, the measurements are often skewed by the variations in diameter, and therefore the pressure exerted by the mucous on the catheter, changing the density of the capillary network without changing the blood flow, and changing due to the perfusion measurement. Alternatively, the muscles may relax, which can also affect the measurement. These variations are specific to individuals and their occurrence is difficult to anticipate.

Furthermore, for a urethral tissue, the instrumentation needs to be paired with a good evaluation of the perfusion, and a reduced congestion due to the size of the urethra.

### BRIEF SUMMARY OF THE INVENTION

A goal of the invention is to provide a perfusion monitoring system making it possible to consider the variations in the diameter of the tubular tissue, particularly the corresponding pressure variations in the evaluation of the perfusion.

To achieve this, the invention offers a perfusion monitoring system of a tissue, including a perfusion sensor, a pressure sensor placed near or on the perfusion sensor, the said sensors being connected to a treatment unit.

According to a first aspect, the treatment unit includes at least one correcting module configured to identify and/or correct the artifact of a perfusion sensor connected to a pressure measured by the pressure sensor.

Advantageously, the system according to the invention includes a corrector module that makes it possible to account for the pressure variations connected to the mucous diameter variations in the evaluation of the perfusion. The measurement of the perfusion is thus more specific despite the pressure variations of the individual-specific muscular relaxation.

According to other aspects taken alone or combined according to all the possible technical combinations:

The said corrector module is or includes a module to apply a corrector parameter to the signal coming from the perfusion sensor based on the signal received by the pressure sensor; and/or

The system preferably includes a deployment mechanism placed close to or on the perfusion sensor, the deployment mechanism is configured to apply pressure on the tissue and the said corrector module is or includes a module to determine the counterpressure to apply on the tissue based on the signal received by the pressure sensor, and/or apply the said counterpressure; and/or

The system includes a tubular catheter body equipped with a perfusion sensor and a pressure sensor, and the deployment mechanism includes a stint inside the body or an inflatable deployment balloon extended over a part of the body's section; and/or

The system includes the said deployment balloon and the pressure sensor is radially placed inside the perfusion sensor; and/or

The deployment balloon extends on part of the body section; and/or

The system includes the said stint, and the perfusion sensor and the pressure sensor are diametrically opposite on the body; and/or

The system includes a maintenance balloon at one end of the body; and/or

The perfusion sensor is a photo-plethysmography sensor.

Furthermore, the invention has a procedure to monitor the perfusion of a tissue through a catheter, including the steps to:

Monitor a perfusion of the tissue at the catheter,

Monitor the pressure that the tissue applies on the catheter,

Correct the perfusion monitoring artifacts based on the monitoring of the pressure applied.

According to other aspects taken alone or combined according to all the possible technical combinations:

The correction step includes a step to apply a corrector parameter to the signal coming from the perfusion monitoring based on the pressure monitoring applied.

The correction step includes a step to determine a counterpressure to apply on the tissue based on the monitoring of the pressure applied, and/or apply the said counterpressure.

Another purpose of the invention regards a device to monitor the perfusion of a tissue, particularly a catheter, including a tubular body equipped with a perfusion sensor configured to detect a tissue perfusion, and a deployment mechanism near or on the perfusion sensor, the deployment mechanism being mobile between a deployment position in which it locally increases the size of the body and a collapsed position in which it does not locally increase the size of the body, characterized in that the deployment mechanism includes a stint inside the body or an inflatable deployment balloon extending over a part of the body section.

Advantageously, the device according to the invention would be very useful for urethral tissue, because it includes a specific instrumentation connected to a reduced congestion of the deployment mechanism, while allowing urine to pass through the tubular body.

### BRIEF DESCRIPTION OF THE SEVERAL VIEWS OF THE DRAWINGS

The invention will be explained in further detail by the description of the non-exhaustive modes of embodiment and based on the figures annexed.

FIG. 1 is a schematic view of a general diagram of the system, according to a variant of the invention.

FIG. 2 is a schematic view of a diagram of the treatment unit of a system according to a variant of the invention.

FIG. 2A is a graph illustration of a coefficient curve k based on the pressure.

FIG. 3 is a detailed schematic view of a diagram of a system based on a first variant of the invention.

FIG. 4 is a detailed schematic view of a diagram of a system according to a second variant of the invention.

FIG. 5A is a detailed schematic view of a diagram of a deployment mechanism according to a second alternative of the second variant.

FIG. 5B is a transversal sectional view of a cut in the middle of FIG. 6A.

FIG. 6 is a detailed schematic view of a diagram of a deployment mechanism according to a first alternative of the second variant.

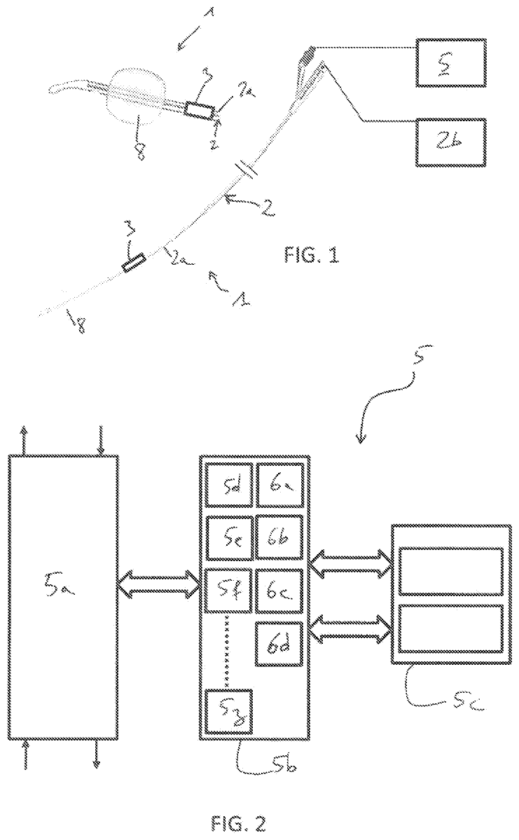

### DETAILED DESCRIPTION OF THE INVENTION

This invention regards a perfusion monitoring system 1 of a tissue like a urethral mucous. In particular the system 1 includes a probe in the form of a catheter 2. The catheter 2 includes a body 2a, preferably a tubular one.

The system 1 includes a perfusion sensor 3 configured to detect a tissue perfusion. The perfusion sensor 3 is, for example, a sensor that uses photo-plethysmography technology, namely a photo-plethysmography sensor. This type of sensor 3 connected to such a system 1 and measuring the related perfusion were presented in detail, particularly in requests WO2013127819 and WO2015044336, regarding the measurements in a particular tissue, namely duodenal mucous.

For a photo-plethysmography sensor to give a reliable indication over time, there must be a constant trail of photons between the issuer and receiver in a given application. In particular, the sensor must be flat against the wall, secondly the interface between the sensor and the mucous wall must always be the same, and thirdly, the composition of the tissue must remain unchanged.

In the event of a urethral measurement, the first point is resolved by the very nature of the urethra, which physiologically collapses in order to block the return of urine. If a catheter 2 is set up, the urethra collapses in the same way on the catheter 2.

Regarding the second point, it is also solved by continuously emptying the bladder using a tubular catheter.

The third point is the most often verified point on a case-by-case basis. The diameter of the urethra and therefore the pressure exerted by the mucous on the sensor can vary on a case-by-case basis. Likewise, during a measurement, the muscles may relax depending on the general state of the tissue.

In both of these cases, the density of the capillary network of the mucous monitored by the perfusion sensor 3 has changed, while the flow in the organ has not increased. This results in a visible variation of the blood flow, which is meaningless since the perfusion has not been actually modified. The perfusion measurements may therefore be skewed by artifacts related to the contraction and relaxation of the tissue mucous, particularly the urethral mucous.

In the aim to simplify this, maximum relaxation is considered, in the invention's interpretation, as no pressure measured, particularly when there is no contact between the sensor and the wall.

To take these artifacts into consideration, the invention offers a pressure sensor 4 in the system 1, particularly on the catheter 2. More particularly, the pressure sensor 4 is placed close to or on the perfusion sensor 3. This arrangement makes it possible to better estimate the pressure applied to the perfusion sensor 3 representing an effort to graft the tissue onto the perfusion sensor 3. Preferably, the pressure sensor 4 is reasonably placed on the perfusion sensor 3, particularly in reference to a longitudinal axis of the catheter body 2a, for more precision. For example, the pressure sensor 4 can be radially placed inside the perfusion sensor 3, or diametrically opposed as shown in more detail below.

The perfusion sensor 3 and the pressure sensor 4 are configured to be associated to a treatment unit 5. The treatment unit 5 makes it possible to treat the signals perceived by the perfusion sensor 3 and the pressure sensor 4. The treatment unit 5 includes for example a computer.

Preferably, the catheter 2 is also connected to a urine receptor 2b receiving the urine evacuated from the bladder through a tubular catheter 2a body. A “Y” connector can be used for this at the end of the catheter 2a body.

According to one aspect, the treatment unit 5 includes at least one corrector module 6 configured to determine and/or correct the perfusion sensor artifacts 3 connected to a pressure measured by the pressure sensor 4.

The treatment unit 5 can be a computer including material means and computer means. In particular, the treatment unit 5 includes material elements such as a connection module 5a, for example a connection BUS, a control module 5b, for example a CPU, and a memory module 5c. Furthermore, the treatment unit 5 includes computer modules 5d, 5e, [ . . . ], 5z, and at least one module 6a, 6b, 6c, 6d. The computer modules 5d, 5e, [ . . . ], 5z, are known modules that make it possible to operate the treatment unit 5, for example known computer modules. The computer modules 6a, 6b, 6c, and 6d are variants of the corrector module 6 allowing, depending on the case, to identify the artifacts, propose a correction and to correct them.

The applicant thus needed to make a model of the tissue grafting efforts to analyze their effects on the perfusion. Surprisingly, the applicant observed that a catheter 2 includes a perfusion sensor 3, a deployment mechanism 7, and preferably a pressure sensor 4, close to or on top of one another, making it possible to apply internal pressure and replicate the tissue grafting efforts, particularly to analyze the effects of the pressure measured on the perfusion measurement.

The preferred catheter 2 and the deployment mechanism 7 will be explained in further detail.

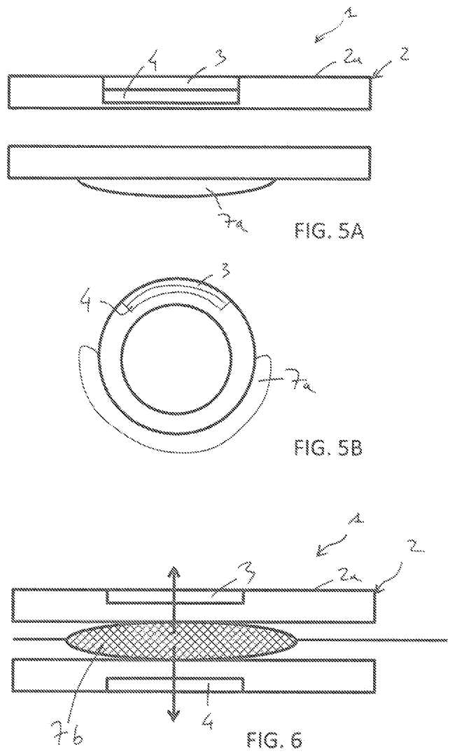

According to one variant, the catheter body 2a is tubular, equipped with a perfusion sensor 3 and a pressure sensor 4, and the deployment mechanism 7 includes an inflatable deployment balloon 7a extending over a part of the body section. This variant can be illustrated by FIGS. 5A and 5B. In particular the deployment balloon 7a extends over half of the body section 2a. It is thus a hemi-balloon. The applicant was surprised to discover that this configuration made it possible to limit the congestion of the catheter 2, which has an effect on small sized tissue, such as that of the urethra.

Preferably, the deployment balloon is a high-pressure balloon. Advantageously a high-pressure balloon is, by its nature, totally collapsed when the balloon is deflated, which makes it possible to insert the probe in the meatus without risking the balloon swathes getting stuck.

Preferably, the pressure sensor 4 and the perfusion sensor 3 are placed on the balloon 7a and the pressure sensor 4 is placed radially inside the perfusion sensor 3. Advantageously, this configuration makes it possible to have specific perfusion measures and pressure applied in the measurement zone.

According to a variant, the catheter 2a body is tubular and equipped with a perfusion sensor 3 and a pressure sensor 4, and the deployment mechanism includes an internal stint 7b at the body. This variation can be illustrated by FIG. 6. The applicant was surprised to discover that this configuration also made it possible to limit the congestion of the catheter 2.

Preferably, the pressure sensor 4 and the perfusion sensor 3 are placed on the stint 7b and are diametrically opposite on the body 2a. Advantageously this configuration also makes it possible to have specific perfusion measurements and applied pressure at the measurement zone.

According to a variant, the monitoring system 1 includes a maintenance balloon 8 at one end of the body 2a. The maintenance balloon 8 makes it possible to keep the catheter body 2a at a fixed position in the urethra. In particular, the maintenance balloon 8 can be positioned in the bladder and be inflated to keep it at the start of the urethra. Depending on the destination of the catheter, the maintenance balloon 8 can be set at a rather large distance from the sensors 3, 4 due to the anatomic difference between men and women.

The catheter 2 equipped with the preferred deployment mechanism 7 can be used to model the grafting efforts.

The perfusion measured through the perfusion sensor 3 can be altered by different grafting efforts, thus creating a corresponding artifact. The grafting efforts can be identified by pressure sensor 4 measurements. The pressure measured can be compensated internally by the catheter 2 through the deployment mechanism 7. The incidence of the grafting pressure on the perfusion measurement can therefore be modeled by applying several points of pressure through the deployment mechanism. This makes it possible to identify a corrective parameter to apply to a perfusion measurement for a given grafting pressure.

In particular, the deployment mechanism is used to get pressure levels going from, for example 0.1 kPa to 6 kPa in steps of 0.2 kPa for a duration of 5s. This is preferably done by going back to the minimal pressure between each step, for example for a duration of 5s. Coming back to a minimum level makes it possible to prevent the tissue getting used to the pressure.

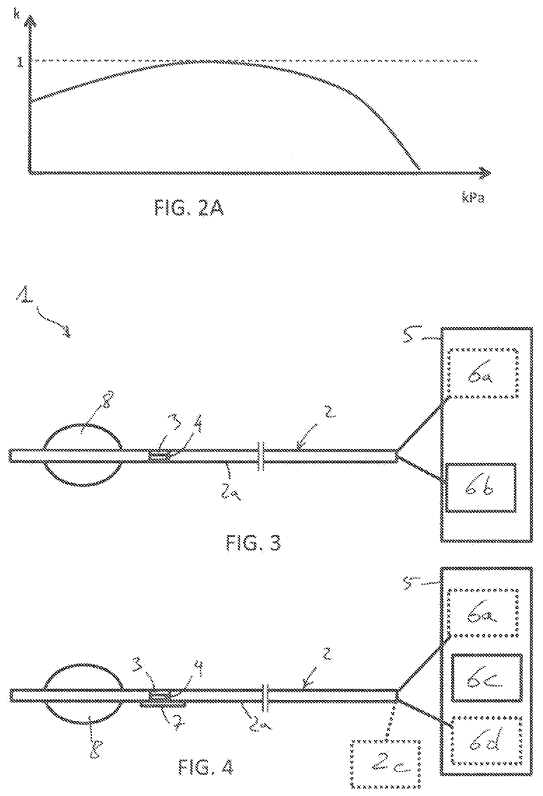

According to the first estimations, the corrective parameter can be a multiplicative coefficient, based on the pressure measured, perhaps obtained, for example as illustrated on FIG. 2A. Preferably, this curve is standardized for its maximum to be 1. Once determined, this coefficient k can be applied to pressure values measured by the pressure sensor 4 to correct the corresponding perfusion measurement, preferably through electronic means. In a simplified example, the perfusion measurement is multiplied by 1/k, with k corresponding to the pressure measurement.

The corrector parameter thus determined can be applied to the systems with or without a deployment mechanism 7 to correct the perfusion measurement. A system variant without a deployment mechanism is illustrated in FIG. 3.

Thus, according to one variant, the treatment unit 5 includes a calibration module 6a making it possible to identify the corrective parameter, particularly in coefficient k. In other terms, the system 1 according to this variant makes it possible to determine, in a given situation with a controlled perfusion and pressure data applied by a deployment mechanism, what the calibration for the corresponding k coefficient is. Thus, it can be calibrated automatically.

Furthermore, according to a variant, the treatment unit 5 includes a multiplicator 6b module connected to a calibration. The calibration can be pre-set with a coefficient k determined for example as described below, logged in the treatment unit memory. In this case, once the calibration has been carried out, the module 6a is no longer necessary in a calibrated monitoring system 1.

Alternatively, the calibration can be done through a calibration module 6a, for example just before the actual measurement. In this case, the module 6a is always connected to the module 6b to redo the calibration.

The module 6b treats the perfusion measurements by correcting them by applying a corrector parameter, in particular the multiplier k coefficient.

Based on the results of the first analyses, the artifacts are connected to a change in the density of the capillary network, itself connected to a variation of the tissue's diameter, which is reflected by a corresponding grafting effort of the tissue on the catheter 2 corresponding to a given effort, maybe compensated by a counterpressure of the catheter 2 on the tissue to reach a target effort.

In particular, the pressure measurements on the catheter 2 make it possible to determine an applied pressure by the tissue on the catheter 2. Thus, it is possible to determine a counterpressure, namely a compensation pressure, making it possible to reach a target effort. If the pressure measured is lower than the target pressure corresponding to a target effort, a counterpressure can be applied by the catheter 2 to reach a target pressure. For example, for a target pressure of 2 in a random unit, making it possible to reach reliable perfusion, if the pressure measured by the catheter 2 is 1, a counterpressure of 1 can be applied by the catheter 2 to offset the measured pressure and reach the target pressure of 2.

Thus, according to a variant, the treatment unit 5 includes an effort determination module 6c. The effort determination module 6c is configured to determine a counterpressure to apply to the tissue, based on the signal received by the pressure sensor 4.

Once the counterpressure is determined, a deployment mechanism, such as an inflatable balloon, preferably the hemi-balloon, or the stint, can make it possible to apply the said counterpressure and have a target grafting effort.

Thus, according to a variant, the monitoring system 1 including the deployment mechanism 7, also includes a means 2c for applying the said counterpressure thus determined. For example, an operator informed of the counterpressure to apply through the effort determination module 6c can apply the said counterpressure by activating the said means. This could be, for instance, a stint command or a remote-controlled inflatable balloon.

In particular, the deployment mechanism 7 is placed close to or preferably on the perfusion sensor 3 in order to get an applied pressure where the pressure is measured.

Alternatively or in combination, the treatment unit 5 can include an effort applier module 6d configured to apply the counterpressure determined by the effort determination module 6c. In particular, the module 6d transmits an order to automatically regulate the pressure of the deployment mechanism 7. Advantageously, this variant makes it possible to automatically offset the pressure without, in this case, needing an operator. A system 1 including both the means 2c for remote control and the module 6d for automatic offsetting, can be provided.

Furthermore, the invention concerns a procedure to monitor the perfusion of a tissue, in particular the tissue of a urethra, through a catheter 2, in particular the catheter 2 as described above.

The procedure according to the invention includes a step to monitor a perfusion of the tissue on the catheter 2, as well as a step to monitor a pressure applied by the tissue on the catheter 2.

According to an aspect, the invention includes a step to correct the artifacts of the perfusion monitoring based on the monitoring of the applied pressure.

According to a variant, the step to correct includes a step for applying a corrective parameter at the signal coming from the perfusion sensor 3 based on the applied pressure monitoring.

According to a variant, the correction step includes a step for determining a counterpressure to apply to the tissue based on the monitoring pressure applied, and/or apply the said counterpressure.

In particular, the procedure makes at least one of the modules 6a-6d described above occur and/or the means 2c for applying pressure.

The monitoring procedure is, in particular, implemented via computer means. Thus, another purpose of the invention regards a product-software or a chargeable computer program in a treatment unit, including portions of the software code to execute the steps of the procedure described above, when the said program is executed on the said treatment unit 5.

Furthermore, each of the modules 6a-6d is in particular a part of the corresponding computer system, for example an electronic monitoring and/or order system. Thus, the invention also concerns a product-software or a chargeable computer program in a treatment unit, including portions of software code to implement at least one of the modules 6a-6d described above, when the said program is executed on the said treatment unit 5.

1. A system for monitoring the perfusion of a tissue, comprising:
a perfusion sensor; and
a pressure sensor disposed near or at the perfusion sensor, wherein said perfusion sensor and said pressure sensor are paired with a treatment unit, and wherein said treatment unit comprises at least one correcting module to determine and/or correct the artifacts of the perfusion sensor linked to a pressure measured by the pressure sensor.

2. The system according to claim 1, in which the said correcting module is or comprises a module for applying a correcting parameter after the perfusion sensor sends a signal based on the signal received by the pressure sensor.

3. The system according to claim 1, further comprising a deployment mechanism disposed near or at the perfusion sensor, the deployment mechanism being configured to apply pressure to the tissue, in which the said correcting module is or comprises a module to determine a counterpressure to apply on the tissue based on the signal received by the pressure sensor, and/or apply the said counterpressure.

4. The system according to claim 3, further comprising a tubular catheter body equipped with a perfusion sensor and a pressure sensor, wherein the deployment mechanism is comprised of an internal stint to the body or an inflatable deployment balloon extending over a part of the body section.

5. The system according to claim 4, further comprising said deployment balloon, in which the pressure sensor is radially disposed in the perfusion sensor.

6. The system according to claim 4, wherein the deployment balloon extends over a part of the body section.

7. The system according to claim 4, further comprising: the said stint, in which the perfusion sensor and the pressure sensor are diametrically opposite on the body.

8. The system according to claim 4, further comprising a maintenance balloon at the end of the body.

9. The system according to claim 1, wherein the perfusion sensor is a photo-plethysmography sensor.

10. A method for monitoring the perfusion of a tissue through a catheter, comprising the following steps:
monitoring a perfusion of the tissue at the catheter,
monitoring pressure applied by the tissue on the catheter, and
correcting artifacts of the monitoring of the perfusion based on the monitoring of the applied pressure.

11. The method, according to claim 10, in which the correction step comprises a step applying a correcting parameter based on the signal from the perfusion monitoring of the applied pressure.

12. The method, according to claim 10, in which the correcting step comprises a step identifying the counterpressure to apply on the tissue based on the applied pressure monitoring, and/or apply the said counterpressure.
